(19) United States
(12) Patent Application Publication
(10) Pub. No.: US 2006/0121099 A1
(43) Pub. Date: Jun. 8, 2006

(54) Process for making nonwoven articles

(75) Inventor: Daniel B. Solarek, Hillsborough, NJ (US)

Correspondence Address: NATIONAL STARCH AND CHEMICAL COMPANY, P.O. BOX 6500, BRIDGEWATER, NJ, 08807-3300, US

(73) Assignee: National Starch and Chemical Investment Holding Corporation, New Castle, NJ (US)

(21) Appl. No.: 11/276,191

(22) Filed: Feb. 17, 2006

Related U.S. Application Data
(63) Continuation-in-part of application No. 10/639,845, filed on Aug. 13, 2003.
Application No. 10/639,845 is a continuation-in-part of application No. 10/219,548, filed on Aug. 15, 2002, now Pat. No. 6,989,339.

(51) Int. Cl. A61K 9/70; A61F 13/15; B32B 27/04

(52) U.S. Cl. 424/443; 442/128; 604/367

(57) ABSTRACT

Method for preparing an active-containing nonwoven article involving fixing active ingredients on the surface of non-woven webs and cellulosic mats. The active ingredients can be released (deposited) onto a surface by normal triggering mechanisms, which include the action of a liquid or by friction or rubbing. The fixative systems described herein provide the ability to control high load levels while not interfering with wettability of the fabric or paper. Additionally, ingredients incompatible and reactive with each other can be treated onto the same web and kept as separate particles fixed to the surface until released.

### CROSS-REFERENCE TO RELATED APPLICATIONS

This application is a continuation-in-part of U.S. application Ser. No. 10/639,845, filed 13 Aug. 2003, which is a continuation-in-part of U.S. Pat. No. 6,989,339, filed 15 Aug. 2002.

### BACKGROUND FIELD OF THE INVENTION

1. Technical Field

The present invention is directed towards processes for applying active-containing compositions onto nonwoven webs. More specifically, the present invention is directed towards processes for applying compositions having one or more active ingredients bound in a hydrocolloid matrix onto with nonwoven articles, as well as nonwoven articles containing those compositions. The present invention is also directed towards processes for delivering active ingredients from those nonwoven articles.

2. Background Information

Nonwoven articles are found in every aspect of modern life. Examples of such articles include diapers and adult incontinence devices, personal care items such as bathroom tissue or baby wipes, and household care products such as surface cleansing wipes. Most, if not all, of these items come in contact with human skin during the normal course of their use.

In order to satisfy consumer demand, it is desirable to make those nonwoven items softer, silkier and more pleasant to use without diminishing the imbibition of the nonwoven web, thereby avoiding interference with the intended utility of the article. For all these products, including household care products, it is desirable to load the web or nonwoven article with an active in an amount that serves the function of the nonwoven article. For example, in diapers it is known to coat a lotion (the active here) onto the topsheet of a disposable diaper (or other nonwoven article). When this nonwoven article is used by the consumer, the lotion is transferred to the wearer's skin, thereby reducing adherence of bowel movements while affording the wearer enhanced skin softness.

Challenges in manufacturing such articles include, for example, applying and maintaining the lotion at or near the surface of the nonwoven web where it will be available for deposition onto a substrate or surface. Typically, actives (e.g., lotions or emollients used in the treatment of skin) are highly mobile materials capable of easily migrating through a web. Migration of actives can have a number of detrimental effects depending upon the type of nonwoven product and its intended application. For example, in the case of the lotion-coated diapers mentioned above, migration of the active lotion away from the surface of the nonwoven renders it no longer available for deposition. Lotion migration can also adversely affect the absorption/transmittance of moisture (e.g., urine) into or through the supporting web of the diapers, interfering with adhesion of the adhesive tabs.

In addition to diapers, examples of other types of nonwoven articles include wipes for personal care applications such as bath and facial tissues, skin care wipes, and so forth, as well as wipes for cleaning hard surfaces such as countertops, floors and automobiles. Like the diaper, each non-woven can be loaded with an active that serves in cleaning or treating the surface.

One method of addressing migration problems of the active involves use of a semi-solid lotion formulation that melts at or around body temperature. This formulation utilizes synthetic waxes and low molecular weight ethoxylates to provide a semi-solid consistency of the formulation. The semi-solid state prevents the lotion active from migrating until the formulation is warmed to body temperature, when it liquefies and deposits onto the skin.

In addition to those problems associated with active migration, high loading of active(s) (i.e., the amount of lotion or emollient applied to a nonwoven web) can have a detrimental effect upon the nonwoven article. For example, active high loading can result in loss of tensile strength and/or reduction in caliper or thickness of the nonwoven sheet. Thickness of the nonwoven is typically correlated to softness and imbibition in the industry (i.e., the thicker the nonwoven, the softer and more absorbent it is). However, active high loading can be a desirable attribute in nonwoven applications.

Therefore, there is still a need for a composition and/or formulation with one or more active ingredients that can be applied to a nonwoven web such that high loading of the active(s) in and/or on the web is provided, while maintaining good softness and feel without interfering with water penetration/absorption or change in article thickness. Further, there is a need for nonwoven webs that provide high loading of one or more active ingredients. Ideally, high loading of these nonwoven webs should be undetectable to the casual user (unless intentionally made to stand out) while still delivering the active to the surface. This active delivery can occur by action of a trigger (e.g., temperature, pressure, friction or an aqueous medium such as body fluids or water) or other trigger release mechanism.

### SUMMARY OF THE INVENTION

In order to address the above issues, the present invention provides a solution for high load fixing or adhering of liquid and semi-solid mobile materials or actives such as fragrances, emollients, cleansing compounds such as surfactants, and skin care lotions to a non-woven web. The actives can be deposited onto a surface such as skin or a countertop by action of a trigger (e.g., water, temperature, pressure and/or friction) while affording minimal interference with the feel or imbibition of the web. This is accomplished in the present invention through the use of compositions having one or more selected starches in combination and compatible with one or more actives.

The process by which the starch/active formulation is applied to the sheet can control the positioning of the ingredients on the nonwoven web or article (e.g., at the surface or in the interior of the web). The formulation can also be applied to specific areas of the web (e.g., at the center of the web or in stripes along the surface of the web) thereby avoiding interference with adhesion or other physical attributes of the nonwoven article. Further, the formulation can be applied to the web in any of a variety of forms (e.g., liquid, mist or powder).

In accordance with the present invention, the following definitions are used—

“Active” as used herein refers to any oily mobile material (e.g., emollient, fragrance, skin care lotion or surfactant) that provides a desired benefit, such as disinfecting a surface, cleansing a surface, adding a moisturizer or other personal care product to skin and/or hair, etc.

“Anhydrous borax fluidity” (‘ABF’) refers to the units that the viscosity of dextrins is typically measured in. The ABF value is defined as the ratio of the amount of water to the amount of anhydrous dextrin when the latter is cooked for 5 minutes at 90° C. with 15% borax (on weight of the dextrin), so as to provide a dispersion having a viscosity of 70 mPas when cooled to 25° C. (see, e.g., U.S. Pat. No. 3,445,838).

“Emollient” as used herein refers to semi-solid or liquid material(s) used to provide a moisturizing, soothing feeling to the skin. Typical emollients suitable for this invention can be soluble or insoluble in water, and preferably are non-volatile under condition of application and use to ensure a durable effect.

“Fixed” refers to the method or process by which a mobile active such as an emollient is held in place in or on the web or the placement of that active onto the nonwoven. The active can be fixed at the surface of the web, internally or both, depending on the specifics of the application.

“Granular starches” refers to any starch (including chemically modified) that is in the same physical form as found in nature (e.g., not swollen or gelatinized).

“High amylose” refers to any starch or flour containing at least about 40% by weight amylose.

“Maltodextrins” refer to purified, concentrated, non-sweet nutritive mixtures of saccharide polymers obtained by partial hydrolysis of edible starch (Food Chemicals Codex, IV Edition, p. 239). Maltodextrins are generally low molecular weight versions of a base starch, whereas pyrodextrins have undergone some level of molecular rearrangement.

“Nonwoven web(s)” refers to any article or sheet-like form made from natural and/or synthetic fibers wherein the fibers are aligned in a random or semi-random order (i.e., not deliberately ordered). One skilled in the art understands that formation of some order occurs during the web forming process (primarily in the machine direction); however, this is completely different from the ordering obtained from traditional weaving or knitting processes. Suitable fibers for use in forming the web include, but are not limited to, cellulose, modified cellulose (cellulose acetate), cotton, polyesters, rayon, polyacrylonitrile (PAN), polylactic acid (PLA), polycaprolactone (PCL), polyolefins and bi-component fiber comprising two or more fiber-forming polymers such as polypropylene and polyethylene terphthalate and the like. Included in the definition of non-woven webs suitable for use with this invention are porous films prepared by the action of chemical or mechanical processing (e.g., apertured films). Also included as useful for the purpose of this invention are paper and paper products.

“Paper” refers to sheet-like masses and molded products made from fibrous cellulosic material. This material can be derived from natural sources, synthetics such as polyamides, polyesters, rayon and polyacrylic resins, as well as from mineral fibers such as asbestos and glass. In addition, paper made from combinations of cellulosic and synthetic materials are applicable herein.

“Papermaking” refers to the process of introducing an aqueous slurry of pulp or wood cellulosic fibers onto a screen or similar device in such a manner that the water is removed, thereby forming a sheet of the consolidated fibers, which, upon pressing and drying, can be processed into dry roll or sheet form.

“Pregelatinized starches” refers to starches treated to destroy the granular structure (i.e., loss of birefringence) and swell or disperse in cold water (CWS starches).

“Pyrodextrins” refer to the hydrolysis product of starch treated at high temperature and low moisture content.

“Surfactant” refers to liquid, semi-solid or solid products used to provide compatibility between the finish and coating component in the formulation. Surfactants can also provide emulsification of the emollient and modification of the hydrophobic properties of the fibrous substrate by allowing rapid transport of aqueous liquids.

“Waxy” refers to any starch or flour containing at least about 95% by weight amylopectin.

Accordingly, the present invention provides a method of preparing an active-containing nonwoven article. According to the present invention, the method includes preparing a solution of at least one active-containing material and at least one fixative; spray-drying the fixative active solution, thereby forming an active-containing particulate, and applying the active-containing particulate to the nonwoven article.

In one embodiment the method further includes compacting the active-containing particulate prior to applying it to the nonwoven article. In another embodiment the method includes agglomerating the active-containing particulate prior to applying it to the nonwoven article.

In one embodiment the method further includes moistening the nonwoven article to aid in fixing the active-containing particulate to the nonwoven article. This moistening can occur prior to applying the active-containing particulate to the nonwoven article, or after. The moistener can be water, solvents, or a combination of water and solvents.

In a further embodiment the at least one fixative includes one or more starch fixatives. These one or more starch fixatives can be, for example, one or more converted starches. The one or more converted starches can be, for example, a maltodextrin and/or pyrodextrin.

In a further embodiment at least one of the one or more starch fixatives includes at least one starch modified with a reagent selected from the group consisting of organic acid anhydrides, alkylene oxides, oxidizing agents and combinations thereof. In one aspect, the reagent is an organic acid anhydride. In a further aspect, the organic acid anhydride is octenyl succinic anhydride. In another aspect, the reagent is an oxidizing agent. In a further aspect, the oxidizing agent is sodium hypochloride. In even another aspect, the reagent is an alkylene oxide. In a further aspect, the alkylene oxide is propylene oxide.

In a further embodiment the at least one active containing material further includes least one surfactant. Useful surfactants include, for example, ionic, anionic, cationic, nonionic and zwitteronic surfactants. Such actives include those suitable for cleansing, disinfecting, degreasing, dispersing and so forth. The active can alternatively be, or also contain additional ingredients dissolved or suspended in the oily material (a ‘mixture of materials’), for example antioxidants, vitamins including vitamin E, medications, and the like.

In even a further embodiment the nonwoven article is a personal care nonwoven article. The personal care nonwoven article can be selected from, for example, the group consisting of diapers, feminine napkins, facial tissues, bath tissues and skin care wipes. In another embodiment the nonwoven article is an industrial or household care nonwoven article. Such industrial or household care nonwoven article can be selected from, for example, the group consisting of cleaning wipes, polishing wipes, anti-rust clothes, lubricating wipes, static control wipes, sanitizing wipes and car care cloths.

### DETAILED DESCRIPTION OF THE INVENTION

In general terms, the present invention provides for a formulation that enables delivery of one or more active ingredients to a nonwoven web, nonwoven webs containing such formulations, and methods for producing the formulation and applying it to the web. The formulation includes at least a fixative ingredient or composition and one or more actives.

Fixative—

According to the present invention, the fixative portion of the formulation includes at least one starch component. While it is typical in today's industry to use synthetic polymers to aid in the fixative process, in a preferred embodiment the present invention is substantially free of fixative polymers other than the presently disclosed starch fixatives. Such other ‘fixative polymers’ include, for example, synthetic fixatives, natural and synthetic waxes, and other low molecular weight polymers. Typical sources for starches and flours are cereals, tubers, roots, legumes and fruits. The native source or base can be corn, pea, potato, sweet potato, banana, barley, wheat, rice, sago, amaranth, tapioca, arrowroot, oat, canna, sorghum and waxy or high amylose varieties thereof. While any starch can be useful in the practice of this invention, the base starches are preferably obtained from corn, tapioca, sago and/or potato. Most suitable for use are the waxy versions of these starches.

Starches according to the present invention can be granular or pregelatinized. Also suitable are converted starches (i.e., starches wherein the molecular weight of the base starch has been reduced) derived from any of the base starch mentioned previously. These include, for example, dextrins prepared by hydrolytic action of acid and/or heat, oxidized starches prepared by treatment with oxidants such as sodium hypochlorite or hydrogen peroxide, and fluidity or thin boiling starches prepared by enzymatic conversion or mild acid hydrolysis.

The selected starch component useful in the fixative formulations of this invention can be unmodified (native) or chemically modified starches or blends of various starches. In one aspect the chemically modified starch component includes starch esters and starch ethers. Useful starch esters and/or starch ethers can contain nonionic or ionic groups such as cationic (e.g., tertiary amine and quaternary ammonium groups) or anionic groups. These starch esters and/or ether can also be crosslinked. The most suitable chemical modifications of the starch component involves treatment with organic acid anhydrides (e.g., octenyl succinic anhydride (‘OSA’)), alkylene oxides (e.g., propylene oxide (‘PO’)), and/or oxidizing reagents (e.g., sodium hypochloride). Modified starches of these types and methods for making them are described in Starch: Chemistry and Technology, R. L. Whistler et al., Eds., Chapter X (1984).

One modification of starch especially useful in the present invention is a starch ester prepared from an organic acid anhydride having a hydrophobic group, for example, octenyl or dodecenyl succinic anhydride. In one aspect the hydrophobic group is a hydrocarbon group such as alkyl, alkenyl, aralkyl or aralkenyl having 2 to 22 carbon atoms; in another aspect the hydrocarbon group has 5 to 18 carbon atoms; and even in another aspect the hydrocarbon group has 8 to 12 carbon atoms. Generally the starch can be treated with up to about 60% by weight of anhydride based on weight of starch in forming the starch ester. In another embodiment the starch can be treated with from about 1 to about 60% by weight of anhydride based on weight of starch. In even another embodiment the starch can be treated with from about 3 to about 10% by weight of anhydride based on weight of starch. A detailed description of starch ester synthesis is found in U.S. Pat. Nos. 2,661,349 and 5,672,699.

For the present invention suitable starches can be converted to water fluidity (“WF”) of at least 40. (The higher the WF the lower the molecular weight of the converted starch, and thus the lower the viscosity.) Most suitable are starches converted to water fluidity greater than about 70 (e.g., maltodextrins or pyrodextrins). Water fluidity measurement as described herein is made using a Thomas Rotational Shear-Type Viscometer (manufactured by Arthur H. Thomas Co., Philadelphia, Pa.) in accordance with standard procedures such as disclosed in U.S. Pat. No. 4,499,116. A further detailed description of this measurement is presented infra in the Examples section.

Active—

The active treated or applied onto the web can be one or more active ingredients or a mixture of materials. Since the active can be fixed to the web as a discrete particle, it is possible to fix two or more incompatible or reactive materials to the web using this invention. These incompatible or reactive materials can be applied to the web simultaneously or sequentially. The materials avoid contact with each other even though they are fixed onto the same web, and can be made to interact with each other by action of a triggering mechanism.

Actives useful in the present invention include oily mobile materials such as emollients. Examples of commercially available classes of emollients suitable for use in the present invention include, without limitation, hydrocarbon oils and waxes, acetoglyceride esters, silicone oils, ethoxylated glycerides, triglyceride esters, alkyl and alkenyl esters, fatty acids and alcohols and their esters and ethers, lanolin and its derivatives, waxes derived from natural or synthetic sources, phospholipids and polyhydric alcohol esters. Some common examples include Aloe Vera, petrolatum, mineral oil, essential oils, hydroxy fatty acids, mono-, di- and tri-glycerides, esters and amides of fatty acids and the like. Particularly suitable emollients are mineral oil, petrolatum, vegetable oil, paraffin oil, and silicone oils. The active can also be a blend of one or more emollients and/or surfactants. The active can also contain additional ingredients dissolved or suspended in the oily material (a ‘mixture of materials’), for example antioxidants, vitamins including vitamin E, medications, and the like.

In one embodiment the emollient contains a functional amount of one or more surfactants. Classes of surfactants useful for this invention are listed below. This mixture of emollient and surfactant is typically referred to as the finish. The finish can contain from about 5 to about 90% by weight of emollient, with the remainder being one or more surfactants.

Typically the finish is prepared by heating the solid components until all have melted, stirring until the mixture is homogenous, and then cooling with continuous stirring. The finish can be added to the fixative portion of the coating composition while hot or after cooling and either in undiluted form or as a dilution, usually in water.

In another embodiment, the oily mobile material of this invention is at least one or more surfactants. Useful surfactants include, for example, ionic, anionic, cationic, nonionic and zwitteronic surfactants. Non-limiting examples of surfactants suitable for use in the present invention include sulfonates of (C₁-C₂₂)alkanes and (C₂-C₂₂)alkenes; (C₈-C₂₂)fatty acids of the formula R³COOH, where a mean average R³ is from about 8 to about 22 saturated or unsaturated carbon atoms and salts thereof (e.g., alkali metal, ammonium, lower alkyl amine and lower alkanol amine salts, as well as sodium, potassium, ammonium and triethanolamine); ethoxylates (2-30) of (C₈-C₂₂)fatty amines; polyoxyethylene polyols selected from sorbitol, glycerine, pentaerythritol, trimethylol ethane, trimethylol propane, and neopenyl glycol; sorbitan (C₈-C₂₂) fatty acids; ethoxylated (1-20 moles) sorbitan (C₈-C₂₂) fatty acid esters which are uncapped or capped with (C₁-C₁₀), preferably (C₁-C₄), alkoxylates; polyoxyethylene (2-100) sorbitol (C₈-C₂₂) fatty esters; ethoxylated (C₈-C₂₂) fatty alcohols having an ethylene oxide moiety corresponding to the formula —(OCH₂CH₂)ₘ, wherein m is from about 2 to about 100 moles of ethoxylation where these fatty alcohols can be straight or branched chain alcohols and can be saturated or unsaturated; phosphate and sulfonate esters of (C₈-C₂₂) fatty acids; polyalkylene oxide carboxylic acid esters having from about 8 to about 18 carbon atoms and having a polyethylene oxide moiety corresponding to the formula —(OCH₂CH₂)ₙ, where n is from about 2 to about 20, and further where mono-, di- and tri-esters are included, preferably having from about 12 to about 18 carbon atoms and where n is from about 4 to about 20; sulfonate and phosphate esters of C₁₂-C₁₈ fatty acids; sulfosuccinates; sulfosuccinamates; phenol, naphthyl, phenol (C₁-C₁₂) alkyl and naphthyl (C₁-C₁₂) alkyl sulfonates; castor oil ethoxylates (2-200 moles), and block copolymers of ethylene oxide and propylene oxide having from about 2 to about 100 moles of ethylene oxide and from about 2 to about 50 moles of propylene oxide. In another aspect, the block copolymers have from about 2 to about 50 moles of ethylene oxide. In even another aspect, the block copolymers have from about 2 to about 30 moles of propylene oxide. Examples of suitable ethoxylated fatty alcohols include oleth-, ceteth- or stearyl-2 through oleth-, ceteth- or stearyl-20, which are ethylene glycol ethers of the respective alcohols, wherein the numeric designation indicates the number of ethylene oxide moieties present and other fatty alcohols may include lauryl alcohol and isocetyl alcohol.

In one embodiment surfactants include combinations of two or more of polyoxyethylene (2-20) cetyl, stearyl or laureth alcohol, glycerol monooleate, polyoxyethylene(2-20) sorbitan (C₁₂-C₁₈) esters; and/or sorbitan (C₁₂-C₁₈)fatty acid esters. In even another embodiment the surfactants include combinations of two or more of polyoxyethylene(2) cetyl alcohol, sorbitan palmitate, polyoxyethylene(20) sorbitan monolaurate and glycerol monooleate.

Additional Ingredients—

In addition to the fixative and the active, the formulation can optionally contain other additive ingredients normally found in such systems. Some non-limiting examples of these other ingredients include fragrances, colorants, fillers, essential oils, vitamins, disinfectants, chelating agents (e.g., EDTA, citric acid, and other organic acids), and the like.

Nonwoven Web—

Webs containing such formulations can be used in any of a variety of applications. These include wipes for personal care cleansing (e.g., skin cleansing, teeth whitening or self-tanning wipes), household and industrial hard surface cleansing wipes (e.g., wipes for automobiles, windows, countertops and/or floors), insect repellant wipes, polishing wipes and disinfectant wipes. Other nonwoven applications include paper (e.g., flavor or aroma burst paper), textiles, absorbent products such as feminine care napkins and diapers, etc.

The present invention will find utility with any weight of non-woven web and will depend greatly on the requirements of the particular application. Manufacturing processes for making nonwoven webs are well known in the art. These include, for example, wet-laid, air-laid (dry laid), spunbond, spunlace, meltblown and needle punch. Particularly suitable webs will have a base weight (i.e., the weight of the web before any coating or treatments are applied) of less than about 100 grams per square meter (gsm). In another aspect the webs will have a base weight of less than about 20 gsm.

The amount of active material and other components in the load which makes up the formulation can vary depending upon the end use. Load is defined as the total amount of all ingredients except the fixative in the formulation. In other words, the load is the active material(s) (e.g., surfactant, emollient, etc.) and any other optional additive ingredients in the formulation. The load can comprise from about 10 to 100% by weight of active and from about 0 to 90% of other additive ingredients.

The formulation can comprise on a dry basis from about 15 to 90% by weight of load and from about 85 to 10% by weight of fixative. In another aspect, the formulation includes from about 30 to 85% by weight of load and from about 15 to 70% by weight of fixative. In even another aspect the formulation includes from about 50 to 85% by weight of load and about 15 to 50% by weight of starch. The percentage or amount of load is the anhydrous (dry) weight of the load divided by the anhydrous (dry) weight of the total formulation (e.g., total=load plus fixative), multiplied by 100.

Generally, according to the process of the present invention a dispersion of the fixative is prepared. The various components constituting the load are added to the dispersion. These components can include, for example, emollients, surfactants, fragrances and so forth, depending upon the particular end use/application. In one embodiment, the fixative/load formulation can be prepared by cooking the starch fixative at the desired solids content and then emulsifying the load component into the starch fixative cook. Alternatively, the formulation can be prepared by co-cooking the starch with the load with sufficient shear to form the emulsion. Total desired solids content can be obtained by dilution with water.

The formulation can then be applied to a web by spraying dispersion/emulsion onto the web. The method of applying the formulation (fixative+load) to the web can dictate the upper limit on viscosity, but can also vary based on operating parameters used, such as run speed, application amount and application temperature. One skilled in the art will recognize that excessively high viscosities (e.g., greater than 1,000 mPas) require special provisions beyond what is typically used in commercial manufacture.

In another embodiment, the dispersion can be sprayed dried and then applied to the web in powder form. In one aspect, the web is at least slightly moist (e.g., having a moisture content of 10% or less.) A small amount of moisture in the web can cause the fixative to at least partially swell, becoming just tacky enough to stick or adhere to the nonwoven.

Spray drying allows the dispersion to be co-processed. Co-processing involves subjecting the blend to a spray-cooking or drum-drying process, thereby pregelatinizing the starch. An example of a useful spray-cooking process is the Steam Injection Dual Atomization (“SIDA”) process disclosed in U.S. Pat. Nos. 4,600,472 and 4,280,851. Another useful example is the spray-cooking process known as the “EK Process” disclosed in U.S. Pat. Nos. 5,131,953, 5,188,674, 5,281,432, 5,318,635, 5,435,851 and 5,571,552. The EK Process is a continuous coupled process in which starch slurry is jet-cooked, then conveyed at high temperature to a spray drier and spray dried.

In the SIDA process, a mixture of the granular starch dispersion is cooked or gelatinized in an atomized state. The starch which is to be cooked is injected as a starch slurry through an atomization aperture in the nozzle assembly into the spray of atomized steam so as to heat the starch to a temperature effective to gelatinize the starch. An enclosed chamber surrounds the atomization and heating medium injection apertures and defines a vent aperture positioned to enable the heated spray of starch and active to exit the chamber. The arrangement is such that the lapsed time between passage of the spray of load through the chamber, i.e., from the atomization chamber and through the vent aperture, defines the gelatinization time of the starch. The resulting spray-dried pregelatinized starch comprises uniformly gelatinized starch in the form of indented spheres, with a majority of the granules being whole and unbroken and which swell upon rehydration. Nozzles suitable for use in the preparation of these starches are described in U.S. Pat. No. 4,610,760.

The steam injection/dual atomization process as referred to above may be more particularly described as pregelatinization of the starch by:

a) mixing the starch in an aqueous solvent,

b) atomizing the mixture with an enclosed chamber, and

c) interjecting a heating medium into the atomized mixture in the enclosed chamber to cook the starch, the size and shape of the chamber being effective to maintain the temperature and moisture control of the starch for a period of time sufficient to cook the starch.

A steam injection/single atomization process for cooking and spray-drying starch is disclosed in the U.S. Pat. No. 5,149,799 patent referred to above and comprises:

a) slurrying the starch in an aqueous medium,

b) feeding a stream of the starch slurry at a pressure from about 50 to about 250 psig into an atomizing chamber within a spray nozzle,

c) injecting a heating medium into the atomizing chamber at a pressure from about 50 to about 250 psig,

d) simultaneously cooking and atomizing the starch slurry as the heating medium forces the starch through a vent at the bottom of the chamber, and

e) drying the atomized starch.

It is further noted that blends of the selected cross-linked starches may be used. Flour can also be slurried with the starch(es).

In those aspects where appropriate, small-scale modifications of the SIDA process may be used. One skilled in the art would recognize and know such modifications, an example of which is illustrated infra.

According to the SIDA process, the blend is initially mixed in an aqueous solvent (e.g., a slurry is formed) at the desired solids level and ratio of modified starch to flour. Typically, the desired solids level is between about 25% and about 43% by weight. In another embodiment, the solids level is between about 30 and about 35% by weight. The aqueous mixture is then atomized into an enclosed chamber forming a relatively fine spray that may be uniformly cooked or gelatinized. A heating medium can be interjected into the chamber to cook the material. Atomization of the slurry can be effectuated in a multi-fluid nozzle through which the slurry is conveyed, with steam (in this embodiment, the heating medium) interjected through the nozzle into the atomized material. This atomization process results in gelatinization of the blend.

After gelatinizing the atomized fixative, the gelatinized mixture (fixative+active) can be optionally transferred to a spray tower and dried from about 3% to about 12% moisture content by weight of the dried mixture.

Other spray-drying processes can also be used according to the present invention. After being subjected to the spray-cooking or drum-drying process, the processed material may optionally be agglomerated. Agglomeration may be achieved by methods known in the art, including, for instance, via batch or continuous processing. A particularly useful method of agglomeration involves spraying the material recovered from the spray tower with water until the individual particles adhere to one another. The particles are then dried with heated air to final moisture content of from about 3% to about 12%.

The dried powders can be compacted using any means known in the art. A particularly useful method of compacting is by feeding the powder through a roller compactor, such as a chilsonator. After the initial spray-drying or drum-drying wherein the active is encapsulated, the dried powder is subjected to compact granulation or chilsonation in order to build (increase) particle size. No additional moisture is required for this process. Useful particle ranges of interest include conditions wherein approximately 70% of the particles are within the range of about 700 to 800 microns, with the remaining 30% divided above and below this range. In another embodiment, approximately 70% of the particles are within the range of about 1200 microns, with the remaining 30% divided above and below this range. In another embodiment, approximately 70% of the particles are within the range of about 2000 microns, with the remaining 30% divided above and below this range.

Another useful method of compacting is by extrusion. When extrusion is used, starch can become pregelatinized and compacted during the same process. The particle size of compacted CWS starch powders can be reduced by methods known in the art such as milling. The particle size distribution of the powders can also be optionally narrowed using methods known in the art such as sieving. The roller compaction process can also be combined with milling and sieving processes to again obtain a precise particle size distribution.

Once dried and in powder or particulate form, the particles can then be applied to a nonwoven web by any manner known in the art. For example, the particulates can be applied by mechanical means, electrostatic means, and so forth.

Depending on use, the total amount of formulation (load plus fixative) applied to the web will vary greatly with the desired result. Typically the total anhydrous formulation applied to the web will range from about 0.5% to about 50% based on the weight of the web (dry basis).

One skilled in the art will recognize the utility of this invention in applications such as diapers, feminine napkins, skin care wipes, facial and bath tissue, adult incontinence products such as protective underwear, underpads, bladder control beds, and the like. Many other industrial applications may also find utility. Some non-limiting examples are anti-rust wrapping material, fragrance release papers and household or industrial cleaning, polishing, lubricating, sanitizing and absorbent cloths or papers.

### EXAMPLES

The following examples are presented to further illustrate and explain the present invention and should not be taken as limiting in any regard. Unless stated otherwise, all percents are in a weight/weight basis.

Water Fluidity Measurement

Starch water fluidity (‘WF’) is measured using a Thomas Rotational Shear-Type Viscometer (manufactured by Arthur H. Thomas Co., Philadelphia, Pa. 19106), standardized at 30° C. with a standard oil having a viscosity of 24.73 mPas, requiring 23.12+/−0.05 seconds for 100 revolutions. Accurate and reproducible measurements of WF are obtained by determining the time which elapses for 100 revolutions at different solids levels depending on the starch's degree of conversion (as the degree of conversion increases, WF increases and viscosity decreases). The procedure used involves slurrying the required amount of starch (e.g., 6.16 g, dry basis) in 100 ml of distilled water in a covered copper cup and heating the slurry in a boiling water bath for 30 minutes with occasional stirring. The starch dispersion is then brought to the final weight (e.g., 107 g) with distilled water. The time required for 100 revolutions of the resultant dispersion at 81-83° C. is recorded and converted to a water fluidity number using a conversion table.

Time required for 100 Revolutions (seconds)
Amount of Starch Used (anhydrous, g):

| | | | | Water |
|---|---|---|---|---|
| 6.16^{a} | 8.80^{b} | 11.44^{c} | 13.20^{d} | Fluidity |
| 60.0 | | | | 5 |
| 39.6 | | | | 10 |
| 29.3 | | | | 15 |
| 22.6 | | | | 20 |
| 20.2 | | | | 25 |
| | 33.4 | | | 30 |
| | 27.4 | | | 35 |
| | 22.5 | | | 40 |
| | | 32.5 | | 45 |
| | | 26.8 | | 50 |
| | | 22.0 | | 55 |
| | | | 24.2 | 60 |
| | | | 19.2 | 65 |
| | | | 15.9 | 70 |
| | | | 13.5 | 75 |
| | | | 11.5 | 80 |
| | | | 10.0 | 85 |
| | | | 9.0 | 90 |

For ^{a}, ^{b}, ^{c} and ^{d}, final weight of each starch solutions is 107, 110, 113 and 115 g, respectively.

### Example 1

### Dextrin Fixative for Water Insoluble Emollient and Fragrance

This illustrates the production of an emollient emulsion, the spray application of that emulsion on a web and the utility of that treated web.

A pyrodextrin produced from tapioca starch with an ABF of about 4 and that had been treated with about 3% octenyl succinic anhydride was slurried in water and cooked by direct steam injection in a model C-1 jetcooker (National Starch and Chemical Company, Bridgewater, N.J.) to produce a dextrin dispersion at about 45 percent anhydrous solids. About 300 ml of this dispersion at 49° C. (120° F.) was placed into a one liter 316 stainless steel beaker and mixed with a Silverson model L4RT laboratory emulsifier (Silverson Machines, Inc., East Longmeadow, Mass.) fitted with a 31.75 mm (1.25 inch) diameter fine screen emulsifying head. The mixer speed was set at 10,000 rpm. Sufficient Dow Corning 245 silicone oil (Dow Corning, Midland, Mich.) was slowly added over a five minute period to give an anhydrous ratio of 45 parts dextrin and 25 parts Dow Corning oil. Aloe Vera extract (Verogel 1:1, Dr. Madis Laboratories, South Hackensack, N.J.) was added to the dispersion with mixing in an amount sufficient to give an anhydrous ratio of 45 parts dextrin, 25 parts silicone oil and 25 parts aloe extract. Peppermint oil (redistilled peppermint oil FFC obtained from Ungerer Co., Lincoln Park, N.J.) was added to the dispersion with mixing in an amount sufficient to give an anhydrous ratio of 45 parts dextrin, 25 parts silicone oil, 25 parts aloe extract and 5 parts peppermint oil. This was diluted with warm water to about 20% solids.

This dispersion (designated A in Table 1 below) was applied to a 30.48 cm (12 inch) by 40.64 cm (16 inch) portion of polypropylene lightweight diaper topsheet (spunbond/melt blown/spunbond SMS by Polymer Group, Inc., a/k/a PGI, Mooresville, N.C.). The web was placed onto a screen on top of a spray box fitted with air exhaust fans and air drawn through at low velocity to control overspray. The dispersion was sprayed onto the web using a Schlick model 970/4 with 0.5 mm liquid insert (available from Orthos Liquid Systems, Bluffton, S.C.) air atomizing nozzle with an air pressure of about 1.05 kg/cm² (15 psig). The dispersion was pumped to the nozzle through a Masterflex peristaltic pump (available from Cole Parmer Instrument Co., Vernon Hills, Ill.) at about 10 ml per minute. The nozzle, mounted on a wand, was moved over the web, at a distance of about 25.4 cm (10 inches), to apply a uniform treatment to the web. The web was dried in a forced air oven for 1 minute at 65° C. (150° F.), weighed and the dry treatment weight recorded.

The resulting webs were little changed in appearance compared to the starting material. Close or microscopic examination showed the treatment to be present as dried particles fixed on the fibers of the web. The particles were bound to the web and not dislodged by folding, stacking or storage of the web. The web had little or no odor. Vigorous rubbing of the web treated surface or between two other sheets ruptured the particles and released the fixed additives as detected by the oily feel on the fingers or to sudden odor of the peppermint. Water applied to the treated web dissolved the particles and released the fixed additives.

### Example 2

### Visual Determination of Fixative Location of Non-Woven Web

A dextrin dispersion was made using the process described in Example 1. Sufficient Dow Corning 245 silicone oil (Dow Corning, Midland, Mich.) was slowly added over a five minute period to give an anhydrous ratio of 45 parts dextrin and 45 parts Dow Corning oil. Sufficient citric acid (ACS reagent grade, Aldrich Chemical Co, Milwaukee, Wis.) was added (as a 35% solids water solution) to give a ratio of anhydrous components of 45 parts dextrin, 45 parts Dow Corning 245 oil and 10 parts citric acid. Warm water was added to adjust the total solids to about 20%. Red food color dye was added to give a red color to the dispersion for easy visual determination when apply to the web. The dispersion was sprayed onto the web and the web dried as in Example 1 (web Sample B in Table 1 below).

The resulting webs were pink. Microscopic examination showed the treatment to be present as red dried particles fixed on the fibers of the web. The particles were bound to the web and not dislodged by folding, stacking or storage of the web. Vigorous rubbing of the web treated surface or between two other sheets ruptured the particles and released the fixed oil. Water applied to the treated web dissolved the particles and released the fixed oil and acid.

### Example 3

### Dextrin Fixative for Water Soluble Active Material

A dextrin dispersion was made as described in Example 1. Aloe Vera extract (Verogel 1:1, Dr. Madis Laboratories, South Hackensack, N.J.) was added to the dispersion with mixing in an amount sufficient to give an anhydrous ratio of 70 parts dextrin, 30 parts aloe extract. Warm water was added to give total solids of about 20%. Blue food color dye was added to give a blue color to the dispersion. The dispersion was then sprayed onto the web and the web dried as in described in Example 1 (web Sample C in Table 1 below).

The resulting webs were blue. Microscopic examination showed the treatment to be present as blue dried particles fixed on the fibers of the web. The particles were bound to the web and not dislodged by folding, stacking or storage of the web. Vigorous rubbing of the web treated surface or between two other sheets ruptures the particles and releases the fixed aloe. Water applied to the treated web dissolves the particles and releases the fixed aloe.

### Example 4

### Fixative for Separate, Incompatible Materials

Sample B+C (in Table 1 below) is a web treated with approximately equal volumes of dispersion B and dispersion C applied as two passes onto the same web

The resulting webs were purple to the naked eye. Microscopic examination showed the treatment to be present as separate blue and red particles fixed on the fibers of the web. This shows the ability to separately fix potentially incompatible or reactive additives to the same web.

TABLE 1
Polypropylene Treated Webs

| | | Sheet | Sheet | % |
|---|---|---|---|---|
| Expt. | Web | Weight | Weight | Treatment |
| No. | Sample | (untreated) | (treated) | (dry basis) |
| 1 | A | 1.92 g | 3.13 g | 38.6% |
| 2 | A | 1.92 g | 2.91 g | 34.0% |
| 3 | A | 1.92 g | 3.74 g | 48.6% |
| 4 | A | 1.92 g | 2.74 g | 30.2% |
| 5 | B | 1.92 g | 3.07 g | 37.5% |
| 6 | C | 1.92 g | 2.83 g | 32.2% |
| 7 | B + C | 1.92 g | 4.13 g | 53.5% |

The results in Table 1 show the fixing of an emollient oil, a water-soluble extract and/or fragrance oil onto the surface of a non-woven web. The ingredients are held on the surface with no tendency to migrate into the sheet and can be released on contact with water or by mechanical energy (rubbing). These examples also demonstrate the ability to keep reactive/incompatible materials fixed on the fibers of the web, separated from each other until released.

### Example 5

### Dextrins for Emollient Fixation

This example illustrates production of an emollient emulsion, spray application of that emulsion onto a web, and the utility of that treated web.

A pyrodextrin produced from tapioca starch with ABF of about 4 and that had been treated with about 3% octenyl succinic anhydride was slurried in water and cooked by direct steam injection in a model C-1 jetcooker to produce a dextrin dispersion at about 30 percent anhydrous solids. About 3000 ml of this dispersion, at 65° C. (150° F.), was placed in a 7 liter 316 stainless steel beaker and mixed with a Silverson model L4RT laboratory emulsifier (Silverson Machines, E. Longmeadow Mass.) fitted with a 31.75 mm (1.25 inch) diameter fine screen emulsifying head. The mixer speed was set at 10,000 rpm. Sufficient molten petrolatum as the active ingredient (Sonojell 9 Witco Chemical Corp., Greenwich, Conn.) was slowly added, over a ten minute period, to give an anhydrous ratio of 20 parts dextrin and 80 parts petrolatum. After an additional 15 minutes of mixing the 50% weight median particle size diameter was about 1 micron. This was dyed pink with red food coloring dye and diluted with warm water to about 35% solids.

The dispersion was applied to a polypropylene lightweight diaper topsheet (spunbond/melt blown/spunbond, SMS by PGI, Mooresville, N.C.). A pilot scale continuous hotmelt laminator (Independent Machine Co.) was modified to spray the emulsion. A 35.56 cm (14-inch) wide roll of web was mounted on the supply spindle, and the web was then brought through the tension control rolls horizontally across the converting section. A two fluid, air atomizing, flat fan spray nozzle (Spraying Systems, ¼ J setup SUN 13) was mounted above the web. Emulsion was supplied through tubing to the nozzle from an air-pressurized vessel at 2.1 kg/cm² (30 psig). Atomizing air was supplied to the nozzle at about 6.7 kg/cm² (35 psig). The nozzle was set to treat the central 20-25 cm (8-10 inches) of the web.

The web then passed under a warm air jet and was wound on the take-up roll. Web speed and feed pressure was varied to adjust the emulsion delivery rate to the web. The sample was labeled D and the results are shown in Table 2 below.

A pyrodextrin produced from tapioca starch with an ABF of about 4 and that had been treated with about 3% octenyl succinic anhydride was slurried in water and cooked by direct steam injection in a model C-1 jetcooker to produce a dextrin dispersion at about 30 percent anhydrous solids. About 3000 ml of this dispersion, at 65° C. (150° F.), was placed in a 7 liter 316 stainless steel beaker and mixed with a Silverson model L4RT laboratory emulsifier (Silverson Machines, East Longmeadow, Mass.) fitted with a 31.75 mm (1.25 inch) diameter fine screen emulsifying head. The mixer speed was set at 10,000 rpm. Sufficient molten petrolatum/surfactant mixture was slowly added, over a ten minute to give a dry ratio of 30 parts starch and 70 parts of load (57.1% Petrolatum USP Witco, 14.3% Brij 52, 21.5% Tween 20, 7.1% Span 80; all available from Uniqema, New Castle, Del.). This was dyed blue with blue food coloring dye and diluted with warm water to about 35% solids. This formulation was applied to the web in the fashion described above. The sample was labeled E and the results are shown in Table 2 below.

These treated webs and an untreated control were tested for synthetic urine wet through (European Disposables And Non-wovens Association) non-woven cover stock liquid strike-though time (simulated urine), EDANA test method 150.3-96) with the exception that Whatman #1 was used in place of Hollingsworth & Vose ERT FF3w/s filter paper. The time to penetrate the web is shown in Table 2 as ‘Strike Through’.

TABLE 2
Emollients fixed to a non-woven web with and without surfactant

| | | Petrolatum | Petrolatum | Strike |
|---|---|---|---|---|
| Sample | Surfactant | pickup (%) | (g/M²) | Through (sec) |
| D | No | 18.9 | 2.895 | 136 |
| D | No | 7.7 | 1.184 | 124 |
| Control | No | 0 | 0 | 79 |
| E | Yes | 10.3 | 1.482 | 42 |
| E | Yes | 18.6 | 2.863 | 45 |

This example demonstrates the use of modified dextrins as a fixative for water insoluble emollients onto the surface of a non-woven web. The addition of surfactants to the fixative formulation allows the treated web to transport water (aqueous fluids) at rates similar to untreated sheets while maintaining a high loading of water insoluble emollients. The emollients are released by the action of the water or mechanical forces (rubbing) to be deposited onto the skin.

Other useful surfactant blends which with Capsul TA starch at 30 weight percent and emollient of petrolatum at 70 weight percent (wherein the load of petrolatum/surfactant or surfactant blend is part of the petrolatum component) provided desirable strike-through effect are:
(a) Load: 57.1% Petrolatum USP, available from Witco; 14.3% cetyl alcohol, 21.5% sorbitan monopalmitate (Span 40), 7.1% polyoxyethylene (20) sorbitan monostearate (Tween 60), all available from Uniqema, New Castle, Del.
(b) Load: 57.1% Petrolatum USP, available from Witco; 21.4% glycerol monooleate, 16.1% sorbitan monooleate, 5.4% polyoxyethylene (20) sorbitan monolaurate (Tween 20, available from Uniqema, New Castle, Del.).
(c) Surfactant blend: 50% glycerol monooleate, 37.5% sorbitan monooleate, 12.5% polyoxyethylene (20) sorbitan monolaurate, all available from Uniqema, New Castle, Del. The amount of surfactant blend ranged from 1 to 50 weight percent of the starch/petrolatum weight amount.

### Example 6

### Screening Evaluation for Various Starch-Based Fixatives

Dextrin dispersions were prepared as described in Example 1 and diluted to 20%. The cooked starches were then blended with Atphos® MBA 1310 and polyoxyethylene Lial 125 (C₁₂₋₁₅) alcohol at the specified anhydrous ratio and were blended with a Silverson model L4RT laboratory emulsifier (Silverson Machines, East Longmeadow, Mass.) fitted with a 31.75 mm (1.25 inch) diameter fine screen emulsifying head for about 5 minutes. These mixtures were drawn on a glass plate as a 0.254 mm (0.01 inch) wet film and dried at room temperature for 24 hours.

TABLE 3
screening of various starches for use as emollient fixatives

| | | Starch/ | | |
|---|---|---|---|---|
| | Formulation | Emollient | | |
| Starch | Solids | Ratio | Appearance | Evaluation |
| Waxy maize, | 10% solids | 1:1 | Acceptable | Separate oily |
| WF = 40, | | | Viscosity | film |
| 3% OSA | | | | |
| Waxy maize, | 20% | 1:1 | Acceptable | No separation |
| WF = 70, | | | Viscosity | |
| 3% OSA | | | | |
| Waxy maize, | 20% | 1:1 | Acceptable | No separation |
| WF = 85, | | | Viscosity | |
| 3% OSA | | | | |
| Waxy maize, | 10% solids | 2:3 | Acceptable | Separate oily |
| WF = 40, | | | Viscosity | film |
| 3% OSA | | | | |
| Waxy maize, | 20% | 2:3 | Acceptable | Separate oily |
| WF = 70, | | | Viscosity | film |
| 3% OSA | | | | |
| Waxy maize, | 20% | 2:3 | Acceptable | Slight |
| WF = 85, | | | Viscosity | Separation |
| 3% OSA | | | | |
| Waxy maize, | 10% | 1:1 | Too thick | No separation |
| cross-linked | | | | |
| Potato | 10% | 1:1 | Too thick | Sep oily film |
| Canary corn | 20% | 1:1 | Acceptable | No separation |
| dextrin, | | | Viscosity | |
| ABF = 2 | | | | |
| Canary corn | 20% | 2:3 | Acceptable | Separate oily |
| dextrin, | | | Viscosity | film |
| ABF = 2 | | | | |

“Appearance” shows observations of the wet mixture at specified solids; too thick could not be readily sprayed or roll coated (typically greater than 1000 mPas). This demonstrates that a certain minimum level of conversion (hydrolysis of the base starch or reduction of the molecular weight) is desirable for most applications.

“Evaluation” shows observations of the dry films and predicts the ability of these mixtures to fix the emollient in a dry particle. Oil separation shows that the starch and surfactant are not compatible and will not be able to hold (fix) the emollient onto the surface of the web.

This example also shows that certain starches, while being useful as a fixative for the emollient, may be too viscous to make application practical. Likewise, low viscosity starches may be suitable for application but may not function acceptably in fixing the active onto the web, especially at higher loadings.

### Example 7

### Oily Mobile Formulation Applied to a Paper Surface

A pyrodextrin with an ABF of about 4 produced from tapioca starch that has been treated with about 3% octenyl succinic anhydride is slurried in water and cooked by direct steam injection in a model C-1 jetcooker to produce a dextrin dispersion at about 45 percent anhydrous solids. About 300 ml of this dispersion, at 49° C. (120° F.), is placed in a one liter 316 stainless steel beaker and mixed with a Silverson model L4RT laboratory emulsifier fitted with a 31.75 mm (1.25 inch) diameter fine screen emulsifying head. The mixer speed is set at 10,000 rpm. Sufficient petrolatum as the active is slowly added, over a five minute period, to give an anhydrous ratio of 25 parts dextrin to 75 parts Dow Corning oil. The oily mobile formulation is diluted with warm water to about 20% solids.

This dispersion is applied to tissue paper of a basis weight of about 55 g/m². The web is then placed on a screen on top of a spray box fitted with air exhaust fans and air drawn through at low velocity to control overspray. The dispersion is sprayed onto the web using a Spraying Systems SS1/4J air atomizing nozzle with an air pressure of about 1.05 kg/cm² (15 psig). The dispersion is pumped to the nozzle through a Masterflex peristaltic pump at about 10 ml per minute. The nozzle, mounted on a wand, can be moved over the web, at a distance of about 25 cm (10 inches), to apply a uniform treatment to the web. The web is then dried in a forced air oven for 1 minute at 65° C. (150° F.).

The above example illustrates the utility of the invention for treating cellulosic webs (e.g. paper or fabric) with a fixed emollient system. The emollient will be held in place on the surface of the cellulose mat until released by water or friction and has utility in facial and bath type tissue.

### Example 8

### Oily Mobile Formulation

This example illustrates production of a surfactant-containing composition useful for applying to non-woven webs and its application thereto.

Acid-hydrolyzed amylopectin chemically modified with 1-octenyl butane dioate was added to 0.833 kg of water to form a 2.083 kg 30% solids starch solution. 1.250 kg of a 50% active quaternary ammonium cationic surfactant (commercially available as Barquat® 4250-Z, a quaternary ammonium disinfectant with alkyl dimethyl benzyl ammonium chloride and alkyl dimethyl ethylbenzyl ammonium chloride as the active ingredients, from Lonza Ltd., Basel, Switzerland) was added to the solution. The solution was mixed and heated to 40-45° C. and spray-dried at an inlet nozzle temperature of about 143-166° C. (290-330° F.) and outlet nozzle temperature of about 74-82° C. (165-180° F.). An off-white, yellowish free-flowing powder of 50 parts dextrin to 50 parts surfactant was produced.

The resultant powder can be applied to a moistened non-woven web, causing the starch fixative component to swell and adhere to the web, thereby fixing the co-processed surfactant active to the web. The web can be moistened with water and/or solvent. The nonwoven can then optionally be dried, resulting in an active-containing nonwoven article. The resultant product is useful, for example, as a disinfectant for countertops.

The resultant powder was further chilsonated. Three cuts of the powder centering around 800, 1200 and 2000 microns were taken. The chilsonated powders were then applied to a non-woven web as described above.

### Example 9

### Oily Mobile Formulation

This example illustrates production of a surfactant-containing composition useful for applying to non-woven webs and its application thereto.

3.90 kg of a 100% active nonionic surfactant (commercially available as Triton® DF-12, a low foam nonionic surfactant, from The Dow Chemical Company, used in household cleansing as, for example, a defoamer or degreaser) was added with mixing to 5.60 kg of water. The solution was then added to a 10.0 kg 39% solids starch solution (amylopectin chemically modified with 1-octenyl butane dioate). The solution (40% solids) was mixed and spray-dried at an inlet nozzle temperature of about 191-196° C. (376-385° F.) and outlet nozzle temperature of about 93-99° C. (200-210° F.). An off-white powder of 50 parts modified starch to 50 parts surfactant was produced.

The resultant powder can be applied to a moist non-woven web, causing the starch fixative component to swell and adhere to the web, thereby fixing the co-processed surfactant active to the web. The nonwoven can then optionally be dried, resulting in an active-containing nonwoven article. The resultant product is useful, for example, as a disinfectant for countertops.

The resultant powder was further chilsonated. Three cuts of the powder centering around 800, 1200 and 2000 microns were taken. The chilsonated powders were then applied to a non-woven web as described above.

### Example 10

### Oily Mobile Formulation

This example illustrates production of a surfactant-containing composition useful for applying to non-woven webs and its application thereto.

3.50 kg of a 100% active nonionic surfactant (commercially available as Triton® DF-12, a low foam nonionic surfactant, from The Dow Chemical Company) was added with mixing to 12.70 kg of water. The solution was then added to a 10.0 kg 35% solids starch solution (acid-hydrolyzed amylopectin chemically modified with 1-octenyl butane dioate). The solution (27% solids) was mixed and spray-dried at an inlet nozzle temperature of about 191-196° C. (376-385° F.) and outlet nozzle temperature of about 93-99° C. (200-210° F.). An off-white powder of 50 parts dextrin to 50 parts surfactant was produced.

The resultant powder can be applied to a moist non-woven web, causing the starch fixative component to swell and adhere to the web, thereby fixing the co-processed surfactant active to the web. The nonwoven can then optionally be dried, resulting in an active-containing nonwoven article. The resultant product is useful, for example, as a disinfectant for countertops.

The resultant powder was further chilsonated. Three cuts of the powder centering around 800, 1200 and 2000 microns were taken. The chilsonated powders were then applied to a non-woven web as described above.

### Example 11

### Oily Mobile Formulation

This example illustrates production of a surfactant-containing composition useful for applying to non-woven webs and its application thereto.

Acid-hydrolyzed amylopectin chemically modified with 1-octenyl butane dioate was added to water to form a starch solution. A 50% active alkyl polyglycoside nonionic surfactant (commercially available as APG®, a wetting and dispersing surfactant consisting of a hydrophilic saccharide moiety and a hydrophobic fatty alkyl chain, from Cognis, Düsseldorf, Germany) was added to the solution with mixing to form a 21-27% solids solution. The solution was spray-dried at an inlet nozzle temperature of about 177° C. (350° F.) and outlet nozzle temperature of about 88° C. (190° F.). An off-white, yellowish free-flowing powder of 50 parts dextrin to 50 parts surfactant was produced.

The resultant powder can be applied to a moist non-woven web, causing the starch fixative component to swell and adhere to the web, thereby fixing the co-processed surfactant active to the web. The nonwoven can then optionally be dried, resulting in an active-containing nonwoven article. The resultant product is useful, for example, as a disinfectant for countertops.

The resultant powder was further chilsonated. Three cuts of the powder centering around 800, 1200 and 2000 microns were taken. The chilsonated powders were then applied to a non-woven web as described above.

### Example 12

### Oily Mobile Formulation

This example illustrates production of an enzyme-containing composition useful for applying to non-woven webs and its application thereto.

Acid-hydrolyzed amylopectin chemically modified with 1-octenyl butane dioate was added to water to form a 1.035 kg starch solution of 29% solids. 0.653 kg ethylene diamine tetra acetic acid (‘EDTA’) (commercially available as Sequestrene K4 EDTA bulk, from CIBA-Geigy, Basel, Switzerland) was added to the solution with mixing to form a 35.2% solids solution. The solution was spray-dried at an inlet nozzle temperature of about 200° C. (392° F.) and outlet nozzle temperature of about 120° C. (248° F.). A yellowish free-flowing powder of 50 parts dextrin to 50 parts EDTA was produced.

The resultant powder can be applied to a moist non-woven web, causing the starch fixative component to swell and adhere to the web, thereby fixing the co-processed surfactant active to the web. The nonwoven can then optionally be dried, resulting in an active-containing nonwoven article. The resultant product is useful, for example, as a disinfectant for countertops.

The resultant powder was further chilsonated. Three cuts of the powder centering around 800, 1200 and 2000 microns were taken. The chilsonated powders were then applied to a non-woven web as described above.

### Example 13

### Oily Mobile Formulation

This example illustrates production of a surfactant-containing composition useful for applying to non-woven webs and its application thereto.

Amylopectin chemically modified with 1-octenyl butane dioate was added to water to form a starch solution. An alkyl diphenyloxide disulfonate anionic surfactant (commercially available as Dowfax 2A1®, useful in cleaning, from The Dow Chemical Company, Midland, Mich.) was added to the solution with mixing to form a solution. The solution was spray-dried at an inlet nozzle temperature of about 191-196° C. (376-385° F.) and outlet nozzle temperature of about 93-99° C. (200-210° F.). An off-white powder of 50 parts dextrin to 50 parts surfactant was produced.

The resultant powder can be applied to a moist non-woven web, causing the starch fixative component to swell and adhere to the web, thereby fixing the co-processed surfactant active to the web. The nonwoven can then optionally be dried, resulting in an active-containing nonwoven article. The resultant product is useful, for example, as a disinfectant for countertops.

The resultant powder was further chilsonated. Three cuts of the powder centering around 800, 1200 and 2000 microns were taken. The chilsonated powders were then applied to a non-woven web as described above.

### Example 14

### Oily Mobile Formulation

This example illustrates production of a surfactant-containing composition useful for applying to non-woven webs and its application thereto.

Acid-hydrolyzed amylopectin chemically modified with 1-octenyl butane dioate was added to water to form a starch solution. A 50% active alkyl dimethyl benzyl ammonium chloride nonionic surfactant (commercially available as Barquat MB-50®, a quaternary ammonium compound useful as a sanitizer or disinfectant, from Lonza Ltd., Basel, Switzerland) was added to the solution with mixing. The solution was spray-dried at an inlet nozzle temperature of about 143-166° C. (290-330° F.) and outlet nozzle temperature of about 74-82° C. (165-180° F.). Off-white, yellowish free-flowing powder of 50 parts dextrin to 50 parts surfactant was produced.

The resultant powder can be applied to a moist nonwoven web, causing the starch fixative component to swell and adhere to the web, thereby fixing the co-processed surfactant active to the web. The nonwoven can then optionally be dried, resulting in an active-containing nonwoven article. The resultant product is useful, for example, as a disinfectant for countertops.

The resultant powder was further chilsonated. Three cuts of the powder centering around 800, 1200 and 2000 microns were taken. The chilsonated powders were then applied to a non-woven web as described above.

Although the present invention has been described and illustrated in detail, it is to be understood that the same is by way of illustration and example only, and is not to be taken as a limitation. The spirit and scope of the present invention are to be limited only by the terms of any claims presented hereafter.

What is claimed is:

1. A method of preparing an active-containing nonwoven article, the method comprising:
preparing a solution of at least one active-containing material and at least one fixative;
spray-drying the fixative active solution, thereby forming an active-containing particulate, and
applying the active-containing particulate to the nonwoven article.

2. The method of claim 1 further comprising the step of compacting the active-containing particulate prior to applying it to the nonwoven article.

3. The method of claim 1 further comprising the step of agglomerating the active-containing particulate prior to applying it to the nonwoven article

4. The method of claim 1 further comprising the step of moistening the nonwoven article to aid in fixing the active-containing particulate to the nonwoven article.

5. The method of claim 4 wherein the nonwoven article is moistened prior to applying the active-containing particulate to the nonwoven article.

6. The method of claim 1 wherein the at least one fixative further comprises one or more starch fixatives.

7. The method of claim 6 wherein at least one of the one or more starch fixatives is one or more converted starches.

8. The method of claim 7 wherein the one or more converted starches is a maltodextrin and/or pyrodextrin.

9. The method of claim 6 wherein at least one of the one or more starch fixatives further comprises at least one starch modified with a reagent selected from the group consisting of organic acid anhydrides, alkylene oxides, oxidizing agents and combinations thereof.

10. The method of claim 9 wherein the reagent is an organic acid anhydride.

11. The method of claim 10 wherein the organic acid anhydride is octenyl succinic anhydride.

12. The method of claim 9 wherein the reagent is an oxidizing agent.

13. The method of claim 12 wherein the oxidizing agent is sodium hypochloride.

14. The method of claim 9 wherein the reagent is an alkylene oxide.

15. The method of claim 14 wherein the alkylene oxide is propylene oxide.

16. The method of claim 1 wherein the at least one active containing material further comprises at least one surfactant.

17. The method of claim 1 wherein the nonwoven article is a personal care nonwoven article.

18. The method of claim 17 wherein the personal care nonwoven article is selected from the group consisting of diapers, feminine napkins, facial tissues, bath tissues and skin care wipes.

19. The method of claim 1 wherein the nonwoven article is an industrial or household care nonwoven article.

20. The method of claim 19 wherein the industrial or household care nonwoven article is selected from the group consisting of cleaning wipes, polishing wipes, anti-rust clothes, lubricating wipes, static control wipes, sanitizing wipes and car care cloths.
